SEQUENCE LISTING (31 sequences)

SEQ ID NO: 1 - DNA, 19 bp, Artificial Sequence
  Synthetic
agcagtagggaatcttcca

SEQ ID NO: 2 - DNA, 17 bp, Artificial Sequence
  Synthetic
caccgctacacatggag

SEQ ID NO: 3 - DNA, 24 bp, Artificial Sequence
  Synthetic
cccttattgttagttgccatcatt

SEQ ID NO: 4 - DNA, 21 bp, Artificial Sequence
  Synthetic
actcgttgtacttcccattgt

SEQ ID NO: 5 - DNA, 25 bp, Artificial Sequence
  Synthetic
cattgacgttacccgcagaagaagc

SEQ ID NO: 6 - DNA, 21 bp, Artificial Sequence
  Synthetic
ctctacgagactcaagcttgc

SEQ ID NO: 7 - DNA, 24 bp, Artificial Sequence
  Synthetic
ggccgtgttgaacgtggtcaaatc

SEQ ID NO: 8 - DNA, 25 bp, Artificial Sequence
  Synthetic
tnaccatttcagtaccttctggtaa

SEQ ID NO: 9 - DNA, 24 bp, Artificial Sequence
  Synthetic
gggaaaccggggctaataccggat

SEQ ID NO: 10 - DNA, 17 bp, Artificial Sequence
  Synthetic
gtcaccttagagtgccc

SEQ ID NO: 11 - DNA, 18 bp, Artificial Sequence
  Synthetic
gaagcttgcttctttgct

SEQ ID NO: 12 - DNA, 19 bp, Artificial Sequence
  Synthetic
gagcccggggatttcacat

SEQ ID NO: 13 - DNA, 20 bp, Artificial Sequence
  Synthetic
acactggatgatctcagtgg

SEQ ID NO: 14 - DNA, 20 bp, Artificial Sequence
  Synthetic
ctgaatccccctccattatg

SEQ ID NO: 15 - DNA, 21 bp, Artificial Sequence
  Synthetic
ccatgacaacggacagcagtt

SEQ ID NO: 16 - DNA, 19 bp, Artificial Sequence
  Synthetic
cctgtcaactgagcacttt

SEQ ID NO: 17 - DNA, 18 bp, Artificial Sequence
  Synthetic
agagtttgatymtggctc

SEQ ID NO: 18 - DNA, 36 bp, Artificial Sequence
  Synthetic
aaggaggtggatccarcccakaaaggaggtggatcc

SEQ ID NO: 19 - DNA, 10 bp, Artificial Sequence
  Synthetic
cggcagcgcc

SEQ ID NO: 20 - DNA, 21 bp, Artificial Sequence
  Synthetic
gttttcccagtcacgacgttg

SEQ ID NO: 21 - DNA, 20 bp, Artificial Sequence
  Synthetic
actcctacgggaggcagcag

SEQ ID NO: 22 - DNA, 21 bp, Artificial Sequence
  Synthetic
gtattaccgcggctgctggca

SEQ ID NO: 23 - DNA, 61 bp, Artificial Sequence
  Synthetic
cgcccggggcgcgccccgggcggggcggggggcacggggggactcctacgggaggcagcag

SEQ ID NO: 24 - DNA, 16 bp, Artificial Sequence
  Synthetic
gtaaaacgacggccag

SEQ ID NO: 25 - DNA, 17 bp, Artificial Sequence
  Synthetic
caggaaacagctatgac

SEQ ID NO: 26 - DNA, 20 bp, Artificial Sequence
  Synthetic
ggccaatatcattggtggta

SEQ ID NO: 27 - DNA, 22 bp, Artificial Sequence
  Synthetic
attgattatgcaagtggtagcc

SEQ ID NO: 28 - DNA, 29 bp, Artificial Sequence
  Synthetic
acttgtggcaaacattcctgctctgttga

SEQ ID NO: 29 - DNA, 21 bp, Artificial Sequence
  Synthetic
ggattagataccctggtagtc

SEQ ID NO: 30 - DNA, 16 bp, Artificial Sequence
  Synthetic
taccttgttacgactt

SEQ ID NO: 31 - DNA, 22 bp, Artificial Sequence
  Synthetic
tgacgggcggtgtgtacaaggc

(19) United States
(12) Patent Application Publication
(10) Pub. No.: US 2014/0134144 A1
(43) Pub. Date: May 15, 2014

(54) USE OF PROBIOTIC BACTERIAL STRAINS AS A PROPHYLACTIC TOOL AGAINST UTERINE INFECTIONS IN PREGNANT FEMALE RUMINANTS

(71) Applicants: Burim Ametaj, Edmonton (CA); Michael Gaenzle, Edmonton (CA)

(72) Inventors: Burim Ametaj, Edmonton (CA); Michael Gaenzle, Edmonton (CA)

(21) Appl. No.: 13/942,534

(22) Filed: Jul. 15, 2013

Related U.S. Application Data
(60) Provisional application No. 61/671,180, filed on Jul. 13, 2012.

(51) Int. Cl. A61K 35/74

(52) U.S. Cl. CPC A61K 35/744; A61K 35/747
USPC 424/93.45; 424/93.4

(57) ABSTRACT

A method of treating uterine infections in a female bovine includes the step of, prior to parturition, intravaginally administering the female bovine with one or more bacterial strains such as Lactobacillus sakei FUA 3089, Pediococcus acidilactici FUA 3140, and Pediococcus acidilactici FUA 3138, effective for treating pathogenic microorganisms associated with postpartum infection of the uterus of female bovines.

### STATEMENT REGARDING SEQUENCE LISTING

The sequence listing associated with this application is provided in text format in lieu of a paper copy and is hereby incorporated by reference into the specification. The name of the text file containing the sequence listing is 48254_Sequence_Final_{—}2013-07-15.txt. The text file is 6 KB; was created on Jul. 15, 2013; and is being submitted via EFS-Web with the filing of the specification.

### FIELD

This relates to the use of bacterial strains as a prophylactic tool in pregnant female ruminants, such as cows.

### BACKGROUND

Recent reports indicate that between 20 to 40% of dairy cows develop uterine infection (i.e. metritis) within the first week of parturition; another 20% develop clinical endometritis within the first month of parturition, and approximately 30% persist into subclinical endometritis. The pathogenic microorganisms associated with postpartum infection of the reproductive tract of cows are Actinomyces pyogenes, Escherichia coli, Fusobacterium necrophorum, and Bacteroides melaminogenicus.

Uterine infections in dairy cows are associated with predisposing factors such as calving difficulty, retained placenta, and the age of the cows, along with the overgrowth of pathogenic microorganisms in the reproductive tract. Immediately after calving, the dilated state of the cervix allows microorganisms from the environment, cow's skin, and fecal material to enter through the vagina into the uterus and initiate inflammation of the endometrium, which is highly associated with infertility. Metritis associated bacteria have been classified as pathogens, potential pathogens, or opportunistic pathogens.

The immediate consequences of uterine infections are presence of pain and distress in affected animals and low milk production and infertility of dairy cows. Indeed cows that have persistent infection of the uterus, in the form of sublinical endometritis or pyometra, fail to remain pregnant and are culled from the dairy herd. Recent data from CanWest DHI Canada (2010) showed that the number one reason for culling of dairy cows is infertility. In fact, almost 30% of all dairy cows culled for sickness were for infertility reasons. The cost of culling cows for infertility, based on the present average market value of a dairy cow (CAN $2,500) and the number of cows culled (i.e. 54,230 cows), is at more than $135 million.

Under normal conditions the vaginal tract of a dairy cow is populated by a diversity of bacteria dominated mainly by lactic acid bacteria (LAB). Recently, it was shown that bacilli and LAB of the genera Enterococcus, Lactobacillus, and Pediococcus were present in the vaginal tracts of both healthy and infected cows. However, the infected cows had more than 1.000-fold increase in the vaginal bacteria population that consisted mainly of E. coli. Moreover, three E. coli isolates harbored the gene coding for Shiga-like-toxin (SLT) I or II. An increasing body of evidence indicates that Lactobacillus strains suppress the growth of other endogenous bacteria in the vagina through the production of organic acids such as lactic acid, H₂O₂, and bacteriocins. The production of organic acids maintains the vaginal pH at acidic values, thereby creating an inhospitable environment for the growth of most endogenous pathogenic bacteria.

### SUMMARY

There is provided a method of using probiotic bacterial strains as a prophylactic tool against uterine infections in a pregnant female ruminant, comprising the step of: prior to parturition, intravaginally administering the female ruminant with a sufficient count of one or more bacterial strains effective for treating pathogenic microorganisms associated with postpartum infection of the uterus of female ruminants.

According to an aspect, the female ruminant may be a female bovine.

According to an aspect, the pathogenic microorganisms may comprise Actinomyces pyogenes, Escherichia coli, Fusobacterium necrophorum, and Bacteroides melaminogenicus.

According to an aspect, the one or more bacterial strains may comprise: Lactobacillus sakei FUA 3089, Pediococcus acidilactici FUA 3140, or Pediococcus acidilactici FUA 3138, alone or in combination.

According to an aspect, the one or more bacterial strains may comprise one or more bacteriocin-producing lactic acid bacterial strains.

According to an aspect, the one or more bacterial strains may comprise a bacterial count of at least 10⁷ cfu, 10⁸ cfu, 10⁹ cfu or 10¹⁰ cfu.

According to an aspect, the method may further comprise the step of administering the one or more bacterial strains at intervals prior to parturition.

According to an aspect, the bacterial strains may only be administered prior to parturition.

According to another aspect, there is provided a method of using probiotic bacterial strains as a prophylactic tool against uterine infections in a pregnant female ruminant, comprising the step of: prior to parturition, intravaginally administering the female ruminant with Lactobacillus sakei FUA 3089, Pediococcus acidilactici FUA 3140, and Pediococcus acidilactici FUA 3138.

According to an aspect, the female ruminant may be a female bovine.

According to an aspect, the one or more bacterial strains may comprise a bacterial count of at least 10⁷ cfu, 10⁸ cfu, 10⁹ cfu or 10¹⁰ cfu.

According to an aspect, the method may further comprise the step of administering the one or more bacterial strains at intervals prior to parturition.

According to an aspect, the bacterial strains may be effective for preventing pathogenic microorganisms from causing postpartum infection of the uterus of female ruminants.

According to an aspect, the bacterial strains may be applied only prior to parturition. The pathogenic microorganisms comprise Actinomyces pyogenes, Escherichia coli, Fusobacterium necrophorum, and Bacteroides melaminogenicus.

According to another aspect, there is provided a method of using probiotic bacterial strains as a prophylactic tool in a pregnant female ruminant, comprising the step of: prior to parturition, intravaginally administering the female ruminant with bacterial strains comprising Lactobacillus sakei FUA 3089, Pediococcus acidilactici FUA 3140, and Pediococcus acidilactici FUA 3138.

According to an aspect, the female ruminant may be a female bovine.

According to an aspect, the one or more bacterial strains may comprise a bacterial count of at least 10⁸ cfu, 10⁹ cfu or 10¹⁰ cfu.

According to an aspect, the method may further comprise the step of administering the bacterial strains at intervals prior to parturition.

According to an aspect, the bacterial strains may be effective for preventing pathogenic microorganisms from causing postpartum infection of the uterus of female ruminant. The pathogenic microorganisms may comprise Actinomyces pyogenes, Escherichia coli, Fusobacterium necrophorum, and Bacteroides melaminogenicus.

According to an aspect, the bacterial strains may be applied only prior to parturition.

According to another aspect, there is provided a method of using probiotic bacterial strains as a prophylactic tool in a pregnant female ruminant, comprising the step of: prior to parturition, intravaginally administering the female ruminant with one or more bacteriocin-producing lactic acid bacterial strains.

According to an aspect, the female ruminant may be a female bovine.

According to an aspect, the one or more bacterial strains may comprise a bacterial count of at least 10⁸ cfu, 10⁹ cfu or 10¹⁰ cfu.

According to an aspect, the method may further comprise the step of administering the bacterial strains at intervals prior to parturition.

According to an aspect, the bacterial strains may be effective for preventing pathogenic microorganisms from causing postpartum infection of the uterus of female ruminants.

According to an aspect, the pathogenic microorganisms may comprise Actinomyces pyogenes, Escherichia coli, Fusobacterium necrophorum, and Bacteroides melaminogenicus.

Other aspects will be apparent from the specification and drawings.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1A depicts the effects of applications on the cervix size.

FIG. 1B depicts the effects of applications on the uterine horn asymmetry.

FIG. 1C depicts the effects of applications on the uterine horn fluctuation.

FIG. 2 depicts the effects of applications on the haptoglobin in plasma.

FIG. 3A depicts the effects of applications on the likelihood of multiparous cows to remain pregnant.

FIG. 3B depicts the effects of applications on the overall pregnancy rate of primiparous cows.

FIGS. 4A and 4B depict panels with the sample results for their PCR detection in E. coli isolates.

FIGS. 5A and 5B depicts images related to the deferred inhibition assay for bacteriocin production.

FIG. 6 is a chart comparing pre-partum samples and post-partum samples of target groups.

### DETAILED DESCRIPTION

There will now be described a method of preventing uterine infections in female ruminants, and more specifically cows using bacterial strains as a prophylactic tool. The method was developed with respect to dairy cows, however it will be understood that the approach described herein will be applicable to other female ruminants.

During pregnancy, the uterus of a female ruminant is generally sterile. After parturition, the dilated state of the cervix immediately after calving allows microorganisms from the environment, skin, and fecal material to enter through the vagina into the uterus once the uterus is open, which may result in uterine infections, or metritis. It has been found that the risk of metritis may be reduced by treating the female ruminant with certain bacterial strains prior to parturition.

Prior to parturition, the female ruminant is intravaginally administered with one or more bacterial strains, such as Lactobacillus sakei FUA 3089, Pediococcus acidilactici FUA 3140, and Pediococcus acidilactici FUA 3138. It has been found that pathogenic microorganisms associated with postpartum infection of the reproductive tract of female bovines, such as Actinomyces pyogenes, Escherichia coli, Fusobacterium necrophorum, and Bacteroides melaminogenicus, are effectively treated when the female bovines are administered with these bacterial strains. Other pathogens may also be present that are treated. The bacterial strains are described in greater detail below.

The effectiveness of certain bacterial strains were tested with respect to dairy cows, as will be described below. It was also found that those cows that were treated with the bacterial strains also experienced a greater rate of uterine involution, as well as greater milk production compared with those cows not treated with the bacterial strains.

The female ruminant is preferably treated with more than one administration prior to parturition. In cows, it was found that two administrations provided acceptable results, but more administrations may also be given. In one example, a bacterial count of at least 10¹⁰ cfu yielded acceptable results. In another example, a bacterial count of about 10⁷ cfu yielded results that were less than optimal, although the samples were also stored for about 1 year, which may have affected their viability. Various administration schedules and bacterial counts, such as 10⁸ or 10⁹, may be determined using routine testing.

Treatment of Female Bovines with Bacterial Strains

There will now be described a study that tested the application of LAB in the vagina of transition dairy cows several times around parturition to lower the incidence of postpartum uterine infections and improve the overall reproductive performance, immune status in dairy cows. In particular, the study was designed to test whether intravaginal administration of a mixture of one isolate of Lactobacillus sakei and two isolates of Pediococcus acidilactici, starting at 2 weeks before the expected day of parturition until 4 weeks after parturition, would lower the incidence of postpartum uterine infections, improve immune status, and enhance the overall pregnancy rate of transition dairy cows. It is believed that these results also support the conclusion that similar results would be had in other female ruminants.

It will be understood that the findings and results described below are specific to the study as described and conducted. Variations from the methods and materials in the study, but that still relate to the treatment of female bovines, may have a material impact on the results achieved. Accordingly, the conclusions drawn below may not be true for every application of the principles related to the treatment of female bovines.

Materials and Methods

Animals, Preparation of Probiotic Bacteria, Treatments, and Diets

The experiment was conducted at the Dairy Research and Technology Centre, University of Alberta. A total of 82 (30 primiparous and 52 multiparous) Holstein dairy cows (overall parity of the calving event that occurred during the study was 2.4±1.5; mean±SD) with an average body condition score of 3.5 were assigned to the present study. All animals were cared for according to the guidelines established by the Canadian Council on Animal Care, and the experimental protocol was approved by the University of Alberta Animal Care and Use Committee for Livestock. Cows (n=82) were randomly allocated into 2 different groups (n=41 cows/group), as control (CTR) and treatment (TRT) group in a completely randomized block design. Cows were blocked by the expected day of calving, parity, BCS at 2 week before the expected day of calving, and milk production on previous lactation. Cows of the TRT group were administered intravaginally once per wk on wk −2 and −1 before the expected day of calving and on wk 1, 2, 3, and 4 after calving with 10¹⁰-10¹² cfu of probiotic/week/cow.

Bacterial cultures were prepared by separately growing each strain of Lactobacillus sakei FUA 3089, Pediococcus acidilactici FUA 3140, and P. acidilactici FUA 3138) in 250 ml of mMRS broth overnight. Bacterial cells were collected by centrifuging at 5,525×g for 20 minutes using the Allegra 25R Centrifuge (Beckman Coulter, Mississauga, Canada). The pellets of all three strains were resuspended and combined in 150 ml of 10% skim milk. Aliquots of 250 μL of the probiotic mixture were made and these samples were freeze-dried at −70° C. using the Freeze Dry System/Freezone 4.5 (Labconco, Kansas City, USA). Prior to treatment, the freeze-dried probiotic mixture was reconstituted in 1 mL sterile 0.9% saline. The control treatment was prepared by making 1 mL aliquots with autoclaved 10% skimmed milk and the samples were stored at −20° C. Both the probiotic and control treatments were administered intravaginally into the cows within 2 h of being taken out of storage at −20° C. Survival of the cultures during strain preparation and storage was monitored by determination of cell counts. Relative to the initial cell count of the overnight cultures, the survival was 73% after freezing and 38% after freeze-drying. No decrease in cell counts was observed during frozen storage of the freeze-dried cells. The three strains were found to be compatible as a mixture and all three strains were recovered from the freeze-dried preparation. Enumeration results showed that 10¹⁰ cells/mL were used for treatments.

Cows of the CTR group received an intravaginal carrier (i.e. 1 mL of reconstituted skimmed milk) once per wk, for 6 wk. A dried mixture of 3 probiotic bacteria (Lactobacillus sakei FUA 3089, Pediococcus acidilactici FUA 3140, and P. acidilactici FUA 3138) that was stored at −20° C. in 3 mL vials was reconstituted in 1 mL sterile 0.9% saline and administered intravaginally to the cows within 2 h. Methodologies for isolation, identification, and growth of probiotic bacteria are described below. The probiotic load was gently deposited through an aseptic manner into the cranio-medial part of the vagina using a sterile insemination pipette and 5 mL plastic syringe. At the start of the experiment, the dry cows were offered a traditional close-up diet, while newly calved cows were switched gradually to lactation TMR within the first 7 d after calving (see Table 1 below). The dietary cationic-anionic balance was considered for multiparous cows. Diets were formulated to meet or exceed daily requirements of dairy cows for NE_{L}, metabolizable protein, fiber, minerals, and vitamins as per NRC (2001) recommendations. Feed samples and orts were taken periodically and were analyzed to assure the daily intake of all pre-designed nutrients.

TABLE 1
Components and chemical composition of total mixed rations
fed to cows during the close up and the lactation period

| Components, g/kg DM | Close-up | Postpartum |
|---|---|---|
| Steam rolled barley grain | 164 | 298 |
| Steam rolled corn grain | 40 | 79 |
| Grass hay | 100 | — |
| Alfalfa hay | — | 96 |
| Alfalfa silage | — | 199 |
| Barley silage | 602 | 203 |
| Dairy supplement¹ | 12 | 125 |
| Animate² | 48 | — |
| Molasses beet sugar | 6 | — |
| Vegetable oil | 7 | — |
| Limestone | 15 | — |
| Vitamin E (5000 IU/kg) | 4 | — |
| Vitamin D₃ (500,000 IU/kg) | 2 | — |

Chemical composition, g/kg DM unless stated

| | | |
|---|---|---|
| DM, % | 437 | 540 |
| NE₁, Mcal/kg DM | 1.55 | 1.71 |
| NDF | 453 | 278 |
| ADF | 267 | 157 |
| NFC³ | 303 | 421 |
| EE | 37 | 46 |
| CP | 147 | 181 |
| Ca | 9.4 | 11.1 |
| P | 4 | 5 |
| K | 19 | 15 |
| Mg | 4 | 4 |

Clinical Observations

All cows were monitored clinically starting at 2 wk before the expected day of calving up to the next successful pregnancy. Clinical records were collected as following: 1) daily monitoring of general appearance (from −2 wk up to +4 wk) and rectal temperature (from −1 wk up to +3 wk) as well as feed intake (from −2 wk up to +8 wk) and milk production during the +8 wk of lactation; 2) veterinary treatments throughout the lactation period; 3) daily monitoring of vaginal discharges and vaginal examination were used to classify the grade of uterine infections. For simplicity, metritis occurring within 21 d postpartum was defined as a severe and mild form only; severe metritis was when cows had watery reddish-brown, purulent, or mucopurulent discharge with or no fetid odor associated with a rectal temperature >39.5° C. and impaired general condition expressed in a lowered feed intake or milk production; a mild form of metritis was defined when cow displayed intermittent, though lesser amount of discharge without a fetid odor and cow did not show an impaired general condition such as lowered feed intake or milk production. The recording of uterine infection occurring after 21 d postpartum were based upon visual observation of presence of abnormal (pus and fetid odor) vaginal discharge at vulvo-vaginal commissure at wk 3 and 5 after parturition. The reproductive tracts were monitored by rectal palpation at wk +3 and +5 to determine delayed uterine involution and endometritis using the following parameters: asymmetry of the uterine horns (at external bifurcation), presence of abnormal fluid or fluctuation of uterine horns, size of the cervix considering a diameter of about >6 cm as abnormal. An experienced veterinarian did all palpations, and premeasured first finger and thumb of the hand of the palpator were used as reference for measuring the width/diameter of the cervix and uterine horns. The herd veterinarian used transrectal ultrasonography on d 32 and 60 post-insemination for pregnancy diagnosis. Ultrasonography of uterus was performed on d 60 post-insemination by the herd veterinarian for diagnosis of pyometra and, if so, then cows were included in the normal clean-up procedure until being inseminated. All breeding records including 1^{st} insemination pregnancy rate, cumulative (1^{st} and 2^{nd}) insemination pregnancy rates, overall pregnancy rates (up to 5 inseminations), early pregnancy losses (between d 32-60 post insemination) and the overall pregnancy losses (d 32 post insemination—calving) were monitored and used to determine the overall reproductive efficiency of the dairy herd.

Feed Intake and Milk Production

Feed intake was measured as a difference between total daily feed given and the total feed refusals in the next morning in each individual cow starting from 14 d before and 50 d after parturition. Milk samples were collected on d 7, 14, 21, and 28 postpartum at 0500 and 1500 h and were analyzed for fat, CP, milk urea nitrogen (MUN), and lactose by mid-infrared spectroscopy at the Central Milk Testing Laboratory (CanWest DHI, Edmonton, Alberta, Canada).

Haptoglobin Analysis

Blood samples were collected from the coccygeal vein shortly before the morning feeding using 10-mL vacutainer tubes (Becton Dickinson, Franklin Lake, N.J.) starting at wk −2, −1, +1, +2, +3, +4, +5, +6, +7, and +8 relative to the date of calving. Blood samples were stored in ice and centrifuged (Rotanta 460 R, Hettich Zentrifugan, Tuttlingen, Germany) within 20 min at 3,000×g and 4° C. for 20 min to separate plasma. All plasma samples were stored at −20° C. until analysis. Concentrations of haptoglobin in the plasma were determined by using commercially available bovine ELISA kits (Tridelta Development Ltd., Greystones, Co. Wicklow, Ireland). According to the manufacturer, the minimum detection limit of the assay was 2.5 mg/mL as defined by the linear range of standard curves. All samples were tested in duplicate, and the optical density at 630 nm was measured on a microplate spectrophotometer (Spectramax 190, Molecular Devices Corporation). Intra-assay coefficient of variation was at <4.0% and inter-assay coefficient of variation was <10%. Sensitivity of the assay is at 0.05 mg/mL.

Statistical Analyses

Data were organized in contingency tables and analyzed using the FREQ and CATMOD procedures of SAS (SAS institute, Cary, N.C.). The model statement contained cow treatment, parity, and their interactions as the main effects. Categorical response variables included incidences of: 1) purulent discharges, 2) foul smelling discharges, 3) abnormal cervix sizes, 4) abnormal uterine horn fluctuations, and 5) abnormal uterine horn symmetry, at 3 or 5 wk postpartum, and overall. A reduced model was used where the two-way interaction was found non-significant. Additional response variables evaluated included, proportion of cows that became pregnant after one or two inseminations and the overall pregnancy rates after <5 inseminations, pregnancy losses at various intervals, incidences of metritis and proportions of cows that required veterinary clean-up before breeding. Haptoglobin and production performance data were analyzed by the MIXED procedure of SAS. The model contained the random effects of cow and block and fixed effects of treatment, week relative to calving, and their two-way interactions. Measurements taken on the same cow at different weeks were considered as repeated measures with an autoregressive 1 variance-covariance structure. Differences in mean responses were considered significant at P<0.05, and the tendency were considered up to 0.05<P<0.10. The rate at which cows became pregnant was examined by survival analysis for multiparous and primiparous cows separately using the JMP software (version 8, SAS institute, Cary, N.C.).

Results

Results of this study showed that the number of cows that developed metritis in the group that was treated with a mixture of 3 LAB was lower compared to the cows in the CTR group (17.1 vs. 51.2%; P<0.01). In addition, data showed that the cases of cows that were included in the cleanup program, as recommended by the farm veterinarian, tended to be lower in comparison with the cows in the CTR group (12.2 vs 26.8%; P=0.06).

Table 1 summarizes the data related to the incidence rates of uterine infections developed in periparturient dairy cows 21 d after parturition. Results indicated that intravaginal treatment with probiotics lowered the overall incidence rates of cows with abnormal purulent discharges (P<0.01). Moreover, the incidence rates of purulent discharges in the two groups were different at 3 wk postpartum (P<0.05) but failed to do so at 5 wk after calving (P>0.05). Furthermore, results presented in Table 1 showed that cows in the treated group had tendencies to have lower incidence rates of foul smelling at 3 wk postpartum (P=0.07) and when considering the overall cases of cows with this abnormality (P=0.08).

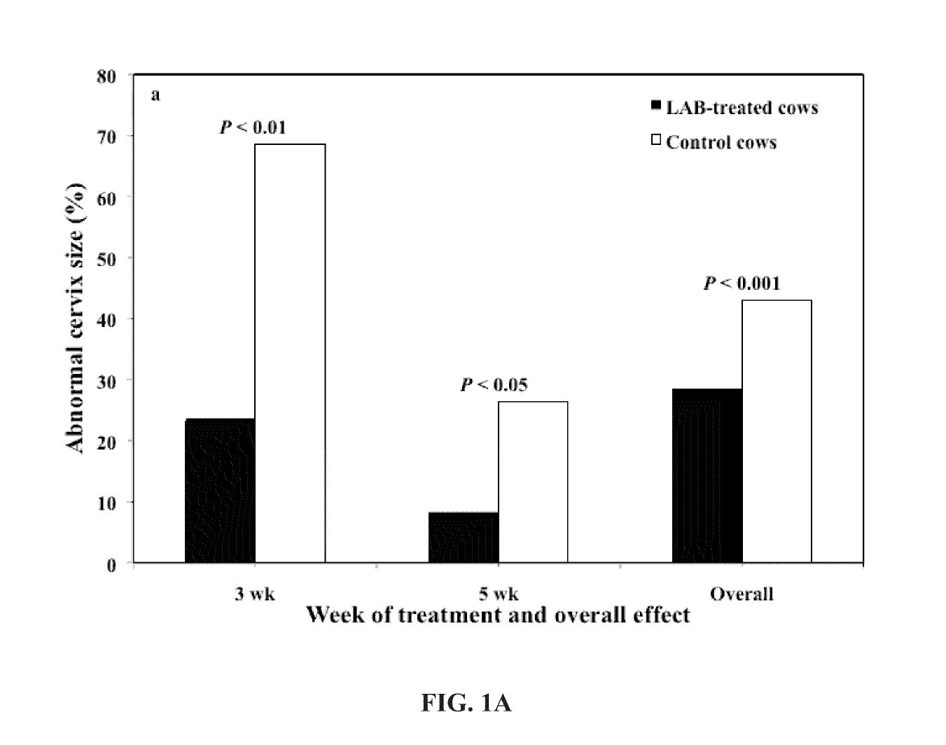

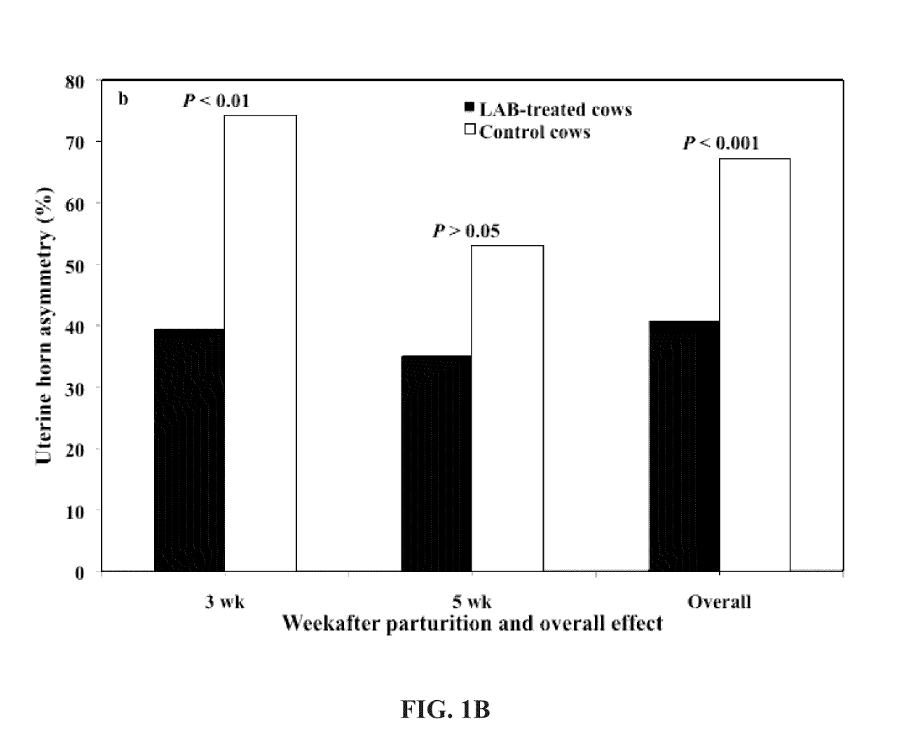

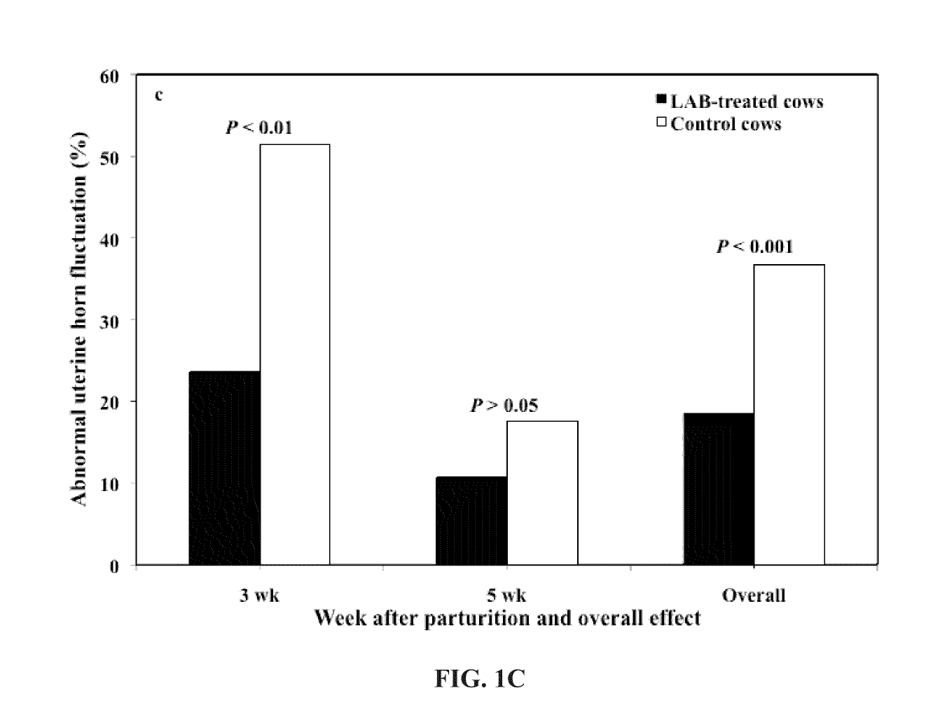

Results related to uterine involution rates of dairy cows in the experiment are shown in FIG. 1A-1C. Intravaginal LAB expedited uterine involution rates in the treated group with cows in the CTR group having greater abnormal cervix size at 3 wk postpartum (P<0.01) and a tendency for greater cervix size at 5 wk postpartum (P=0.06). Referring to FIG. 1A, data showed that 68.6% of the cows in the CTR group versus 22.3% in the TRT group had abnormal cervix size at 3 wk postpartum (P<0.01). In addition, an overall effect of probiotic treatment on cervix size was obtained between the groups (P<0.01).

Referring to FIG. 1B, an overall treatment effect was acquired when relative incidence rates of uterine horn asymmetry data were compared between the two groups indicating that probiotic cows had lower number of cases with asymmetry of the horns compared to the CTR cows (40.8 vs. 67.2%: P<0.05). It was also found that the proportion of cows with horn symmetry was greater in the TRT group of cows at 3 wk postpartum compared to the cows treated with carrier alone (P<0.05); however, no such advantage was observed at 5 wk postpartum despite an apparent numerical difference (35.1 vs. 52.9%; P>0.05).

Referring to FIG. 1C, data also showed that cows receiving intravaginal probiotics had lower overall incidence rates of abnormal uterine horn fluctuations comparative to cows in the CTR group (P<0.05). The advantage of probiotic treatment with regards to uterine horn fluctuations was also obvious at 3 wk postpartum (P<0.05).

The results of this study showed that the reproductive performance of cows was influenced by the probiotic treatment (see Table 2 below). Thus, the overall pregnancy rate indicated a tendency to be greater (P=0.07) in the group of cows treated intravaginally with lactic acid bacteria versus those of the controls. Additionally the pregnancy rates at first insemination and the cumulative insemination rates were numerically greater in the TRT group; however, the difference between the groups did not rich significance (P>0.05).

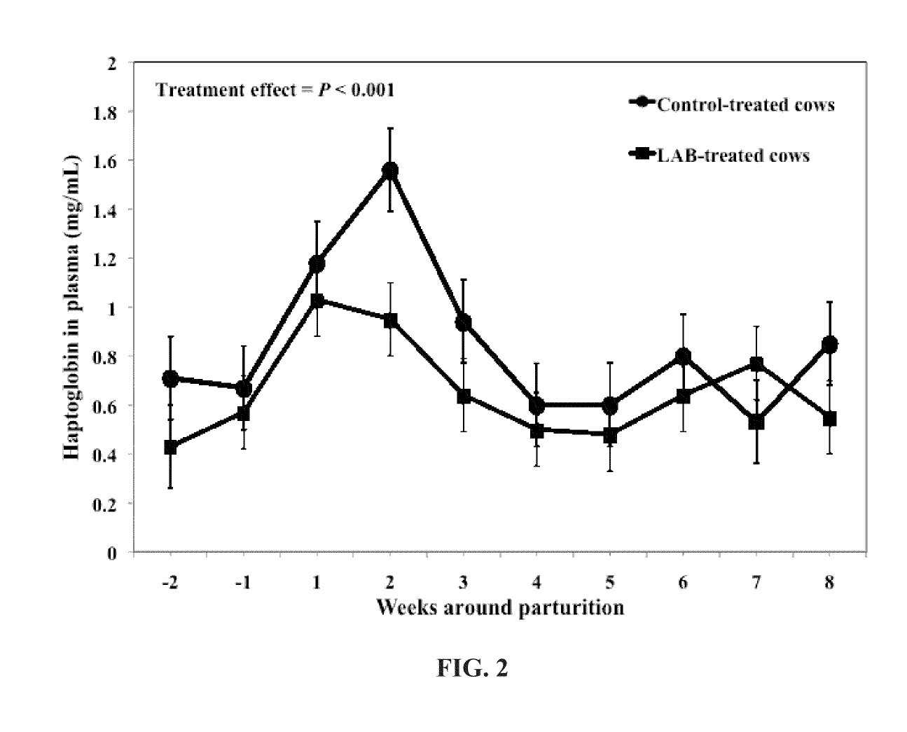

Referring to FIG. 2, concentrations of haptoglobin in the plasma of cows that did not develop uterine infections were lower in the probiotic cows than those in the CTR group (0.656 vs. 0.843 mg/mL; P<0.05). Moreover, although plasma haptoglobin increased in both groups of cows immediately after calving, at 1 and 2 wk postpartum, the peak plasma haptoglobin in the CTR cows was greater than those in the treated ones, especially at 2 wk postpartum (P<0.01).

TABLE 2
The incidence of purulent vaginal discharge and foul
smelling vaginal discharge at 3 and 5 wk postpartum
in periparturient dairy cows treated intravaginally around
calving with a mixture of lactic acid bacteria (LAB)

| | Treatments | |
|---|---|---|
| Item | LAB | Control |
| Incidence of postpartum purulent discharge (%) | | |
| At 3 weeks after parturition | 13.16^{a} | 45.71^{b} |
| At 5 weeks after parturition | 2.78 | 0.00 |
| Overall result | 10.12^{c} | 27.99^{d} |
| Incidence of postpartum foul smelling discharge (%) | | |
| At 3 weeks after parturition | 5.26 | 20.00 |
| At 5 weeks after parturition | 0.00 | 0.00 |
| Overall result | 3.26 | 12.42 |

^{a,b}Within rows, values bearing different superscripts differ at P < 0.05.

TABLE 3
Reproductive performance in periparturient dairy cows
treated intravaginally around calving with a mixture
of lactic acid bacteria (LAB)

Treatment

| Item | LAB | Control |
|---|---|---|
| Overall Pregnancy Rates (%) | 87.88 | 73.53 |
| Pregnancy rates in the first insemination (%) | 36.36 | 29.41 |
| Early pregnancy losses (%) | 3.58 | 7.40 |

¹No significant (P > 0.05) differences were observed.

The analysis of variance indicated that DM intake tended to be greater in the treated multiparous cows (P=0.10), but no difference in DM intake was observed between the control and treated primiparous cows (see Table 4 below). The DM intake was affected by measurement day, whereas there was not treatment by time interaction in this study. Interestingly, the treated multiparous cows produced roughly 3 kg milk more per day than the control counterparts (P=0.01), whereas no difference was observed in the primiparious cows regarding milk yields (Table 4). Milk yield was also affected by sampling day, but not by the treatment by time interaction. Fat percentage and fat:protein ratio in the milk tended to be lower (P=0.07), but lactose content tended to increase (P=0.10) in the multiparous treated cows, whereas no difference was observed in the primiparous cows on all these variables. Also, somatic cell counts in the milk tended to decrease in the treated multiparous cows (P=0.07), but not in the primiparous cows (Table 4). The concentration of milk urea N did not differ between the treatment groups neither in multiparous or primiparous cows.

TABLE 4
Feed intake and milk composition of lactating Holstein cows treated
around calving intravaginally with a mixture of lactic acid bacteria

| | Groups^{‡} | | | P-value^{§} | | |
|---|---|---|---|---|---|---|
| Item^{†} | Control | Treatment | SEM | Trt | D | Trt × D |

Primiparous cows (n = 15)

| | | | | | | |
|---|---|---|---|---|---|---|
| DM intake, kg/d | 14.9 | 15.3 | 0.83 | 0.79 | <0.01 | 0.15 |
| Milk yield, kg/d | 29.8 | 28.6 | 0.60 | 0.16 | <0.01 | 0.81 |
| Fat, % | 4.11 | 4.64 | 0.30 | 0.19 | 0.24 | 0.82 |
| Protein, % | 2.99 | 3.04 | 0.06 | 0.57 | <0.01 | 0.88 |
| Lactose, % | 4.37 | 4.39 | 0.05 | 0.85 | 0.02 | 0.64 |
| Fat:protein ratio | 1.38 | 1.52 | 0.09 | 0.31 | 0.48 | 0.90 |
| SCC, 10³ cells/mL | 238 | 108 | 87.6 | 0.31 | 0.84 | 0.42 |
| Milk urea N, mg/dL | 14.6 | 14.1 | 0.40 | 0.34 | <0.01 | 0.92 |

Multiparous cows (n = 27)

| | | | | | | |
|---|---|---|---|---|---|---|
| DM intake, kg/d | 15.3 | 17.3 | 0.86 | 0.10 | <0.01 | 0.14 |
| Milk yield, kg/d | 34.3 | 37.4 | 0.66 | 0.01 | <0.01 | 0.12 |
| Fat, % | 4.82 | 4.24 | 0.29 | 0.07 | <0.01 | 0.98 |
| Protein, % | 2.87 | 2.85 | 0.05 | 0.82 | <0.01 | 0.03 |
| Lactose, % | 4.26 | 4.37 | 0.04 | 0.10 | 0.86 | 0.19 |
| Fat:protein ratio | 1.69 | 1.49 | 0.07 | 0.07 | 0.08 | 0.74 |
| SCC, 10³ cells/mL | 235 | 73.4 | 58.1 | 0.07 | 0.05 | 0.14 |
| Milk urea N, mg/dL | 14.5 | 14.1 | 0.43 | 0.54 | 0.85 | 0.72 |

^{†}Dry matter intake was measured daily from 14 d before up to 50 d post-parturition, milk yield was measured daily up to d 50 post-parturition; milk samples were taken weekly up to d 50 of the experiment and analyzed for their composition.
^{‡}CTR = no intravaginal administration of lactic acid bacteria; LAB = intravaginal administration of lactic acid bacteria.
^{§}Trt = effect of treatment; D = effect of sampling day; Trt × D = effect of treatment by sampling day interaction.

Discussion

The hypothesis of this study was to evaluate whether intravaginal infusion of a mixture of three vaginal isolates of LAB strains including Lactobacillus sakei FUA 3089, Pediococcus acidilactici FUA 3140, and Pediococcus acidilactici FUA 3138 would be able to lower the incidence of uterine infections, improve immune status, and the overall reproductive and productive performance of dairy cows after parturition. Indeed our data showed that intravaginal treatment of dairy cows around calving with LAB lowered almost 3-fold the incidence rates of severe metritis (during the first 21 d after parturition) in the treated cows. Metritis has been defined as a condition causing systemic signs of illness (e.g. fever, anorexia, and decreased milk production), characterized by a foul-smelling, brown-red, watery vaginal discharge occurring within the first 21 DIM. Among cows with metritis, Escherichia coli and a variety of anaerobic bacteria are common isolates. We reported that cows diagnosed with metritis had greater numbers of E. coli in the vaginal bacterial population. Moreover, 3 of the E. coli isolates harbored the gene coding for Shiga-like-toxin (SLT) I or II. This field trial demonstrates that prophylactic treatment with intravaginal probiotics was able to lower the incidence of metritis in dairy cows. This finding is also in line with research conducted in human subjects showing that intravaginal application of LAB is able to lower the incidence of urinary tract infections; commonly caused by E. coli, Enterococcus fecalis, and Gardnerella vaginalis.

Results of this study also showed that cows in the treatment group had lower incidence of purulent vaginal discharge (PVD) and foul smelling during 3 and 5 wk after parturition. These time points have been used previously to distinguish between metritis and endometritis in dairy cows. In recent investigations it was shown that PVD is related to presence of metritis and endometritis. In fact metritis commonly develops within the first 3 wk after parturition, whereas endometritis usually is diagnosed after the first 3 wk of calving. Recently, it was found that PVD is indicative of clinical endometritis, which commonly affects 15% of the dairy cows after parturition. Indeed cows in this study that developed metritis within the first 21 DIM had also signs of vaginal purulent discharge at 3 and 5 wk after calving. It has also been reported that substantial impairment of reproductive performance of dairy cows affected by clinical endometritis as indicated by extension of days open, decreased overall pregnancy rate, increased involuntary culling, delayed first service, lowered pregnancy to first service, and greater number of inseminations per pregnancy in the affected group of animals.

Administration of probiotics in the vagina of periparturient dairy cows proved beneficial also in relation with the incidence of pyometra. Indeed cows that obtained the LAB treatment had more than 2-fold lower incidence of pyometra. Pyometra is defined as the accumulation of purulent material within the uterine lumen. Pyometra is associated with persistence of corpus luteum, anestrus, and infertility in dairy cows. In addition, the disease requires medical intervention. All the cows in our study diagnosed with pyometra were included in a clean-up program, which adds significant cost to the herd economy.

Another advantageous effect of treating cows with LAB was the lower number of cows with large cervical size and uterine horn asymmetry. Normally both the cervix and the uterus are expected to have diameters of less than 5 cm by 25 d postpartum, although it takes longer for the cervix to involute. One study that considered the relationship between cervix size with the reproductive performance in cows showed that cows with a larger cervix size had decreased risk of pregnancy at the first insemination and increased mean of days open. They also indicated that the optimum time to assess cervical involution was 3 wk postpartum, and that the optimum threshold was 6 cm. In another study, it was confirmed that a cervical diameter of less than 5 cm is not associated with impaired reproduction in cows and that the critical threshold lies between 6 and 7.5 cm. Previous research indicated that infection of the uterus and peripartum disease like metritis, retained placenta, dystocia, and ketosis delay both cervical and uterine involution rates. It is obvious that administration of LAB in this study lowered the incidence of uterine infection and hastened the involution rate of the uterus and the cervix in the treated cows.

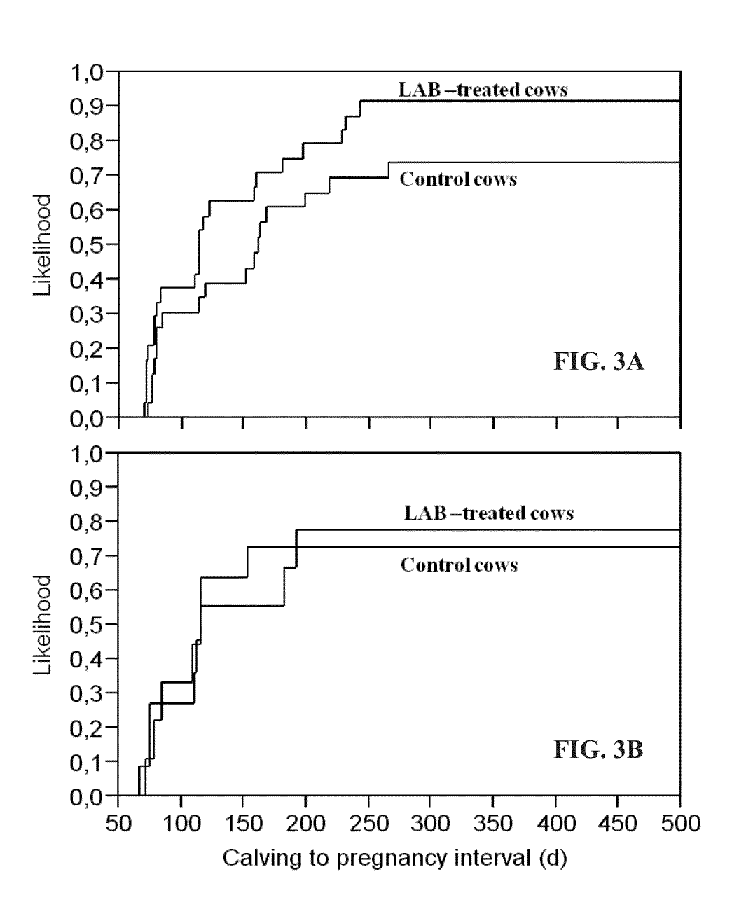

Referring to FIG. 3A, another important finding of this study was that the probiotic treatment improved by more than 20% the likelihood of multiparous cows to remain pregnant. Unexpectedly, referring to FIG. 3B, the treatment had little effect on the overall pregnancy rate of primiparous cows. Although it is not clear what might be the reason for this difference in the results obtained, it should be pointed out that the LAB were isolated from the vaginal tract of healthy multiparous cows. It would be of interest to determine if there are differences in the vaginal microbiota composition between the primiparous and multiparous cows. Also unexpectedly, LAB had no significant effect on the pregnancy rates at first insemination and the cumulative insemination rate although these variables were numerically greater in the TRT group.

Referring again to FIG. 2, haptoglobin measurements in the plasma showed that its concentration was lower at 2 weeks postpartum in cows treated with LAB. Haptoglobin is an acute phase protein that is released several days after initiation of an acute phase response to counteract the effects of bacterial translocation into the host systemic circulation. These results are in agreement with other authors that have reported associations among concentrations of haptoglobin in the blood and infectious diseases of the reproductive tract of dairy cows. Thus, there appears to be a relationship between increased levels of plasma haptoglobin postpartum and the incidence of metritis. Moreover, it has been demonstrated that cows with haptoglobin concentration of more than 1.0 g/L at 3 d after parturition were 7-fold more likely to develop metritis. Additionally, it has been found that blood haptoglobin is a risk factor for reproductive disorders. A cut off concentration of haptoglobin in blood was used, for cows with risk of metritis, at more than 0.8 g/L. It was also shown that concentrations of haptoglobin in blood at more than 0.8 g/L, in the first 7 d postpartum, is associated with more than 2-fold the odds of developing metritis. Therefore lower plasma haptoglobin in the cows treated with LAB is indicative of a better uterine health in those cows.

Evidence suggests various potential mechanisms of action of LAB in protection against uterine infections in the treated cows. Lactic acid bacteria are Gram-positive rods, primarily facultative or strict anaerobes that generally have a particular growth requirement. They have a preference for an acidic environment and support creation of such an environment by producing organic acids, including lactic acid. Lactobacilli have not been associated with disease development and are considered nonpathogenic constituents of the intestinal and urogenital microbiotas. One possible explanation of this is that Lactobacilli compete with pathogenic bacteria by producing biosurfactants that inhibit adhesion of infective bacteria, lower the pH by producing organic acids like lactic acid, release hydrogen peroxide and bacteriocins that inhibit the growth of pathogenic bacteria, and produce coaggregation molecules that block the spread of pathogens.

Feed intake and milk production data showed that multiparous cows treated with LAB intravaginally consumed more dry matter feed and produced more milk than the control counterparts. Given that the probiotics treatment improved the health of the uterus it is reasonable to assume that a healthier cow would eat more and also produce more milk. Cows administered LAB also tended to produce less fat in the milk; however, because those cows produced as an average 3 kg more milk than the control animals the lower milk fat yield was compensated with the greater milk production. Therefore, the overall milk fat production was greater in the treated cows. Moreover, the amount of lactose in the milk was greater in the multiparous treated cows. As expected probiotic treatment had no effect on feed intake and milk production on primiparous cows. As indicated in our discussion, LAB treatment had no effect on uterine health of the primiparous cows either. The mechanism by which LAB affected feed intake and milk production is not clear; however, pain and distress during uterine infections are known to limit feed intake in cows. In addition, previous investigations have shown that lipopolysaccharide released by Gram-negative bacteria during uterine infections translocates into the blood circulation and inhibits feed intake and production of prolactin from the anterior pituitary secretory cells. Prolactin is a hormone released from the pituitary gland that has been proven to have galactopoietic effects in dairy cows.

In conclusion, the results of this study showed for the first time that intravaginal administration of a mixture of two isolates of Pediococcus acidilactici and one strain of Lactobacillus sakei significantly improved postpartum uterine infections and expedited uterine involution in periparturient dairy cows. In addition, data demonstrated that haptoglobin, a protein produced by the liver as part of the acute phase response, was lower in the plasma of cows treated with the LAB mixture indicating better health status of the treated cows. Treatment with LAB also improved the overall pregnancy rate and increased milk production in the multiparous cows but had no effect on the primiparous cows. Milk production and SCC also were lower in the treated cows. Further research is warranted to understand the mechanism(s) involved and to support these findings in a larger cohort of cows as this might have important implication for the dairy industry.

Characterization Of Bacterial Strains

There will now be given a description of a study that characterized the vaginal microbiota of both healthy pregnant and infected postpartum cows by culture-dependent analysis, PCR clone library construction, and quantitative PCR (qPCR). Isolates were studied with regards to Shiga-like toxin and pediocin production.

Methods

Animals

Thirteen lactating Holstein dairy cows were used in our study. Animals were maintained at the Dairy Research and Technology Centre of the University of Alberta in Edmonton, Canada. Metritis or uterine infections were diagnosed on the basis of watery reddish-brown, purulent, or mucopurulent discharges with or without fetid odour. Rectal temperatures of 39.5° C. or higher and impaired general condition as expressed in a lowered feed intake or milk production were also taken into consideration for diagnosis. Ethics approval was obtained from the Animal care and Use Committee for Livestock of the Faculty of Agricultural, Life and Environmental Sciences (University of Alberta protocol #A5070-01).

Samples

Vaginal samples were obtained from seven healthy pregnant cows and eight infected postpartum cows. The vulvar area was thoroughly cleaned with water and then disinfected with 30% (vol/vol) iodine solution (Iosan, WestAgro, Saint Laurent, Canada) prior to sampling. A stainless steel vaginal speculum was gently inserted into the vagina, opened, and a long-handled sterile cotton swab was introduced to obtain a sample from the anterolateral vaginal wall. Each sample was collected in 4 mL of 0.1% (w/v) sterile peptone water with 0.85% (w/v) NaCl and 0.05% (w/v) L-cysteine HCl×H₂O. The cotton swab was moistened by immersion in the peptone water immediately before sampling.

Isolation of Microorganisms

Ten-fold serially diluted samples were plated on Reinforced Clostridial Medium (RCM) with 5% animal blood, Endo agar (Difco, Sparks, USA), and modified MRS (mMRS) agar. Representative colonies from each type of plates were purified by repeated streak-plating until uniform colony morphology was obtained. Isolates from mMRS and RCM with blood were streaked on mMRS agars whereas isolates from Endo plates were streaked on Luria Bertani (LB) agars. Frozen stock cultures of each isolate were prepared from a single colony and stored in 60% glycerol at −70° C.

General Molecular Techniques

General DNA manipulations and agarose gel electrophoresis were performed as known in the art. Chromosomal DNA of isolated strains was extracted from 1 ml cultures using a DNeasy® Blood and Tissue Kit (Qiagen, Mississauga, Canada). Unless otherwise stated, PCR amplifications were performed in GeneAmp® PCR System 9700 (Applied Biosystems, Streetsville, Canada) by using Taq DNA polymerase and deoxynucleoside triphosphates (Invitrogen, Burlington, Canada). The PCR products were purified using the QIAquick PCR purification kit (Qiagen). DNA sequence of each PCR product was compared with 16S rRNA gene sequences of type strains in the Ribosomal Project Database Project II (RDP-II; Michigan State University, East Lansing, USA).

Random Ampled Polymorphic DNA-PCR(RAPD-PCR) Analysis

DNA template was isolated as described above. DAF4 primer was used to generate RAPD patterns for isolates from Endo agar and M13V primer was used for RAPD typing of all other strains (see Table 5 below). The reaction mixture contained 10 μL of 5× Green GoTaq® Reaction Buffer (Promega, San Luis Obispo, USA), 3 μL of 25 mM MgCl₂ (Promega), 150 pmol primer, 1 μL of 10 mmol L¹ dNTP (Invitrogen, Burlington, Canada), 1.5 U GoTaq® DNA Polymerase (Promega), and 1 μL of template DNA suspension or autoclaved water filtered with Milli-Q water purification system as the negative control (Millipore Corporation, Bedford, Mass., United States). The PCR program comprised of an initial denaturation step at 94° C. for 3 minutes, followed by 5 cycles of denaturation, annealing and extension steps at 94° C. for 3 minutes, 35° C. for 5 minutes, and 72° C. for 5 minutes. An additional 32 cycles of denaturation, annealing and extension steps were also performed at 94° C. for 1 minute, 35° C. for 2 minutes, 72° C. for 3 minutes, followed by a final extension step at 72° C. for 7 minutes. RAPD PCR products were electrophoresed in a 1.5% agarose gel with 0.5×TBE buffer (45 mmol L¹ Tris base, 45 mmol L¹ boric acid, 1 mM EDTA, pH 8.0). A 2-log molecular size marker (New England Biolabs, Pickering, Canada) was included on all gels.

Partial 16S ribosomal rRNA gene amplcation and sequencing Clonal isolates were eliminated on the basis of their origin and RAPD patterns, and remaining isolates were identified by partial sequencing of 16S rRNA genes. PCR reaction was performed in a master mix with a final volume of 50 μL containing 1.5 U Taq DNA Polymerase (Invitrogen), 5 μL of 10×PCR Reaction Buffer (Invitrogen), 1.5 μL of 25 mmol L⁻¹ MgCl₂ (Invitrogen), 25 pmol of universal bacterial primers 616V and 630R (Table 5), 1 μL of 10 mmol L¹ dNTP, and 1 μL of template DNA. PCR product was electrophoresed in 1.0% (w/v) agarose gel, with a 2-log ladder (New England Biolabs). All sequencing data were obtained from sequencing services provided by Macrogen (Rockville, USA).

Identcation of E. Coli with Species-Specc PCR and API 20E Test System

PCR amplification of the hypervariable regions of the E. coli 16S rRNA gene used known primers. The PCR reaction mix (final volume 50 μL) consisted of 1.25 U Taq DNA Polymerase (Invitrogen), 5 μL of 10×PCR Reaction Buffer (Invitrogen), 1.5 μL of 25 mmol L⁻¹ MgCl₂ (Invitrogen), 100 pmol of ECP79F and ECP620R (Table 5), 1 μL of 10 mmol L⁻¹ dNTP, and 1.5 μL of template DNA. Reference strains used as positive and negative controls are listed in Table 6. The API 20E test system (bioMérieux, Saint Laurent, Canada) was used to confirm identification to the species level. PCR-based detection of Shiga-like toxin producing E. coli (STEC) was conducted with 50 μL reaction mixes that contained 1.25 U Taq DNA Polymerase (Invitrogen), 5 μL of 10×PCR Reaction Buffer (Invitrogen), 1.5 μL of 25 mmol L⁻¹ MgCl₂ (Invitrogen), 1 μL of 10 mmol L⁻¹ dNTP (Invitrogen), 25 pmol SLTI-F and SLTI-R (Table 5), or 25 pmol SLTII-F and 25 pmol SLTII-R. Positive controls are listed in Table 6.

TABLE 5
Primers used in the study

| | | | Annealing |
|---|---|---|---|
| | | (SEQ ID | Temperature |
| Target/Specificity | Primer/Probe Sequence (5′→3′) | NO:) | (° C.) |
| ^{†}Lactobacillus- | Lac1: AGC AGT AGG GAA TCT TCC A | 1 | 62 |
| Pediococcus- | Lab667r: CAC CGC TAC ACA TGG AG | 2 | |
| Leuconostoc- | | | |
| Weissella | | | |
| (Lactobacillus group) | | | |
| (341 bp) | | | |
| ^{†}Enterococcus spp. | Ent-F: CCC TTA TTG TTA GTT GCC ATC ATT | 3 | 60 |
| (144 bp) | Ent-R: ACT CGT TGT ACT TCC CAT TGT | 4 | |
| ^{†}Enterobacteriaceae | Enterobac-F: CAT TGA CGT TAC CCG CAG AAG AAG | 5 | 63 |
| (195 bp) | C | | |
| | Enterobac-R: CTC TAC GAG ACT CAA GCT TGC | 6 | |
| ^{†}Staphylococcus Spp. | TstaG422: GGC CGT GTT GAA CGT GGT CAA ATC | 7 | 55 |
| (370 bp) | TstaG765: TIA CCA TTT CAG TAC CTT CTG GTA A | 8 | |
| ^{†}Bacillus spp. | BacF: GGGAAACCGGGGCTAATACCGGAT | 9 | 55 |
| (995 bp) | BacR: GTC ACC TTA GAG TGC CC | 10 | |
| ^{†}E. Coli | ECP79F: GAA GCT TGC TTC TTT GCT | 11 | 54 |
| (544 bp) | ECP620R: GAG CCC GGG GAT TTC ACA T | 12 | |
| ^{†}SLT-I | VT1 (SLTI-F): ACA CTG GAT GAT CTC AGT GG | 13 | 55 |
| (614 bp) | VT2 (SLTI-R): CTG AAT CCC CCT CCA TTA TG | 14 | |
| ^{†}SLT-II | VT3 (SLTII-F): CCA TGA CAA CGG ACA GCA GTT | 15 | 55 |
| (779 bp) | VT4 (SLTII-R): CCT GTC AAC TGA GCA CTT T | 16 | |
| 16S rDNA | 616V: AGA GTT TGA TYM TGG CTC | 17 | 52 |
| Sequencing | 630R: AAG GAG GTG GAT CCA RCC | 18 | |
| (~1500 bp) | CAKAAAGGAGGTGGATCC | | |
| Random Primer for | DAF4: CGG CAG CGC C | 19 | 35 |
| RAPD | M13V: GTT TTC CCA GTC ACG ACG TTG | 20 | 35 |
| Universal Primers | HDA1: ACT CCT ACG GGA GGC AGC AG | 21 | 52 |
| | HDA2: GTA TTA CCG CGG CTG CTG GCA | 22 | |
| | HDA1 + GC: CGC CCG GGG CGC GCC CCG GGC GGG | 23 | |
| | GCG GGG GGC ACG GGG GGA CTC CTA CGG GAG | | |
| | GCA GCA G | | |
| TA Cloning | M13Forward (−20): GTA AAA CGA CGG CCA G | 24 | 55 |
| | M13Reverse: CAG GAA ACA GCT ATG AC | 25 | |
| ^{†}Pediocin Structural | pedA2RTF: GGC CAA TAT CAT TGG TGG TA | 26 | 60 |
| Gene pedA (100 bp) | pedA2RTR: ATT GAT TAT GCA AGT GGT AGC C | 27 | |
| | TqM-pedA: FAM-ACT TGT GGC AAA CAT TCC TGC | 28 | |
| | TCT GTT GA-TAMRA | | |
| ^{†}Total Bacteria | TotalBac-F785: GGA TTA GAT ACC CTG GTA GTC | 29 | 52 |
| (727 bp) | TotalBac-R1512r: TAC CTT GTT ACG ACT T | 30 | |
| | TaqMan 1400r Probe: 6-FAM-TGA CGG GCG GTG TGT | 31 | |
| | ACA AGG C-TAMRA | | |

TABLE 6
Reference strains used in the study.

| Strain | Description |
|---|---|
| Lactobacillus plantarum FUA3099 | Positive control for RAPD with |
| | M13V primer |
| Shigella boydii ATCC4388 | |
| Shigella dysenteriae ATCC188 | Negative control for species specific |
| | PCR |
| Shigella flexneri ATCC62 | of E. coli 16S rRNA gene |
| E. coli O157:H7 ATCC43888 | Positive control for species specific |
| | PCR of E. coli 16S rRNA gene |
| E. coli O157:H7 ATCC43889 | SLT-II positive control |
| E. coli O157:H7 ATCC43890 | SLT-I positive control |
| Pediococcus acidilactici FUA3072 | Bacteriocin-producing strains |
| | expressing |
| Pediococcus acidilactici FUA3100 | the pediocin AcH/PA-1 operon |
| Lactobacillus sakei FUA3089 | Non-bacteriocinogenic meat isolate |
| Listeria innocua ATCC33090 | Indicator strains used in deferred |
| | inhibition assay for bacteriocins |
| | detection |

Deferred Inhibition Assay for Bacteriocin Detection

Overnight cultures of test strains were prepared in MRS broth that contained 2 g L⁻¹ glucose. Test strains used in this study included Lactobacillus sakei FUA3089, and Ped. acidilactici FUA3072, FUA3138 and FUA3140. MRS plates with 2 g glucose L⁻¹ were spotted with 3 μL of each overnight culture and the plates were incubated overnight under anaerobic conditions at 37° C. Ped. acidilactici FUA3072 was used as a positive control.

Cultures of indicator strains (Table 6) grown in overnight MRS broth with 2 g L⁻¹ glucose were used to inoculate MRS soft agar at an inoculation level of 1% and the soft agar was overlayered over the MRS plates with test strains. Indicator strains included E. coli FUA1036, E. coli FUA1063, E. coli FUA1064, Listeria innocua ATCC33090, and Enterococcus facaelis FUA3141.

The deferred inhibition assay was repeated with the addition of 20 g L⁻¹ proteinase K in 100 mmol L⁻¹Tris-Cl, pH 8.5, which was spotted adjacent to test strain colonies and plates were incubated for four hours at 55° C. to maximize proteinase activity before overlayering was conducted.

Identification of Library Clones Via Sequencing

A clone library was constructed using PCR products that were amplified with HDA primers, which were then cloned into a pCR 2.1-TOPO vector, using the TOPO TA Cloning® Kit (Invitrogen) according to manufacturer's instructions. The Promega's Wizard® Plus SV Minipreps DNA Purification System was used for plasmid isolation. To confirm the cloning of the inserts, sequencing of the amplified insert was performed according to the Invitrogen TOPO TA Cloning® Kit manual.

Quantitative PCR

Quantitative PCR was conducted with vaginal mucus samples collected from ten randomly selected metritic cows, using syringes fitted with an approximately 30 cm long collection tube. Total bacterial DNA was extracted using the Wizard MagneSil® Tfx™ System (Promega) and DNA concentrations were measured using the NanoDrop spectrophotometer system ND-1000, software version 3.3.0 (Thermo Fisher Scientific Inc., Wilmington, USA).

All dagger-marked primer pairs that are listed in Table 5 were used in the preparation of standards and qPCR analyses. Standards were prepared using purified PCR products, which were serially diluted ten-fold. Diluted standards (10⁻³ to 10⁻⁸) were used to generate standard curves. TaqMan probes were used for the pedA gene and the total bacteria qPCR experiments. In both cases, each probe was labeled with 5′-FAM and 3′-TAMRA as fluorescent reporter dye and quencher respectively. The total reaction volume was set to 25 μL, which contained 12.5 μL TaqMan Universal PCR Master Mix (Applied Biosystems), 2.5 μL of template DNA extracted from vaginal mucus and 5 μmol L⁻¹ of each primer (Table 5), and 0.2 μmol L⁻¹ of the TaqMan probe. SYBR green assays were used for all remaining target-group primer pairs. The total reaction was also set at 25 μL containing 12.5 μL Fast SYBR Green Master Mix (Applied Biosystems), 1 μmol L⁻¹ primer, and 1 μL DNA template. Amplification conditions generally followed an initial denaturation at 95° C. for 5 min for 1 cycle; 40 cycles of denaturation at 95° C. for 30 sec, annealing with listed annealing temperatures in Table 5 for 1 min, and extension at 72° C. for 2 min. Quantitative PCR was executed using a 7500 Fast Real-Time PCR System (Applied Biosystems, Foster City, Calif., USA). Reactions were performed in triplicates in MicroAmp Fast Optical 96-well reaction plates, sealed with MicroAmp Optical Adhesive Film (Applied Biosystems).

Results

Composition of Microbiota in Healthy and Infected Dairy Cows: Isolation and Identification of Bacterial Species

Analysis of the microbiota of the reproductive tract of dairy cows was initially based on a qualitative, culture-dependent approach. Bacterial isolates were obtained from healthy, pre-partum animals (n=7) or infected, post-partum animals (n=8). Clonal isolates were eliminated by RAPD-PCR analysis and isolates representing different RAPD profiles were identified on the basis of the sequence of approximately 1400 bp of the 16S rRNA genes. Strain identification to species level was based on 97% or greater sequence homology to type strain. Strains of the species E. coli could not be identified on the basis of 16S rRNA sequences alone because of the high homology of rDNA sequences to closely-related species such as Shigella spp. and Escherichia fergusonii. Classification of all E. coli strains was verified with species-specific PCR and API-20E test strips. The biochemical characteristics of isolates matched properties of E. coli (99.8%) in the API-20E database. The identity of thirty isolates and their origin is listed in Table 7.

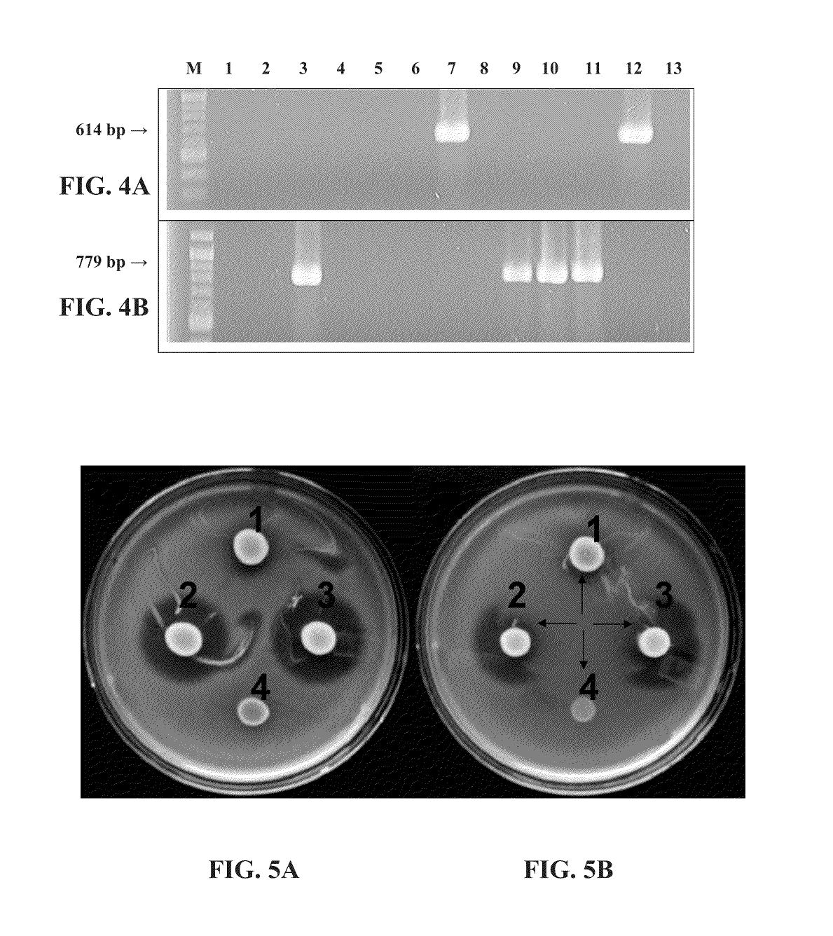

Bacilli, staphylococci, and lactic acid bacteria of the genera Enterococcus, Lactobacillus, and Pediococcus were present in both healthy and infected cows. Escherichia coli was also frequently isolated, particularly from infected animals. Isolates were screened for the presence of SLT-I and SLT-II genes, sample results for their PCR detection in E. coli isolates are shown in FIG. 4A and FIG. 4B, respectively. FIG. 4A depicts a PCR-based detection of shiga-like toxin I (SLT-I)-producing E. coli FUA1064 (lane 7). DNA extracted from E. coli O157:H7 ATCC43890 was used as positive control for SLT-I (lane 12). FIG. 4B depicts a PCR-based detection of SLT-II-producing E. coli FUA1037 (lane 3), and E. coli FUA1062 (lanes 9 and 10). DNA extracted from E. coli O157:H7 ATCC 43889 was used as positive control for SLT-II (lane 11). E. coli FUA1064 isolated from cow #2507 harboured the SLT-I gene, while E. coli FUA1037 and FUA1062, isolated from cow #2373 and #2374, respectively harboured the SLT-II gene (see Table 7).

TABLE 7
Qualitative characterization of the vaginal microbiota of dairy cows.
Healthy, pregnant animals and those diagnosed with post partum
uterine infections at the time of sampling are indicated in brackets.

| | | | | Shiga-like | Pediocin |
|---|---|---|---|---|---|
| | | | | Toxin | Immunity |
| Animal # | FUA # | Identified Species | % Identity to Type Strain^{(a)} | Gene | Gene |
| 2102 (Healthy) | 3086 | Staphylococcus epidermidis | 0.990 | n.d. | n.d. |
| | 3087 | Staphylococcus epidermidis | 0.991 | n.d. | n.d. |
| | 3088 | Staphylococcus warneri | 0.985 | n.d. | n.d. |
| | 3089 | Lactobacillus sakei | 0.986 | n.d. | n.d. |
| 2151 (Healthy) | 1167 | Proteus mirabilis | 0.995 | n.d. | n.d. |
| 2363 (Healthy) | 1035 | Escherichia coli | 0.980 (Shigella flexneri) | − | n.d. |
| | 1037 | Escherichia coli | 0.930 | SLT-II | n.d. |
| | 3137 | Pediococcus acidilactici | 0.990 | n.d. | + |
| | 3140 | Pediococcus acidilactici | 1.000 | n.d. | + |
| | 3141 | Enterococcus faecalis | 0.990 | n.d. | n.d. |
| | 3226 | Pediococcus acidilactici | 0.990 | n.d. | − |
| 2367 (Healthy) | 3136 | Staphylococcus warneri | 0.993 | n.d. | n.d. |
| 2374 (Healthy) | 1062 | Escherichia coli | 0.976 (Shigella flexneri) | SLT-II | n.d. |
| | 2027 | Bacillus licheniformis | 0.982 | n.d. | n.d. |
| | 2028 | Bacillus licheniformis | 0.978 | n.d. | n.d. |
| | 3251 | Streptococcus pluranimalium | 0.990 | n.d. | n.d. |
| 2409 (Healthy) | 1046 | Escherichia coli | 0.978 (Shigella flexneri) | − | n.d. |
| | 3135 | Staphylococcus hominis subsp. hominis | 0.991 | n.d. | n.d. |
| 2426 (Healthy) | 2023 | Bacillus altitudinis | 0.998 | n.d. | n.d. |
| | 2024 | Bacillus pumilus | 0.981 | n.d. | n.d. |
| *2211-A (Infected) | 1036 | Escherichia coli | 0.981 (Shigella flexneri) | − | n.d. |
| | 3139 | Enterococcus faecalis | 0.980 | n.d. | n.d. |
| *2211-B (Infected) | 1174 | Escherichia coli | 0.980 | − | n.d. |
| | 1176 | Escherichia coli | 0.980 | − | n.d. |
| | 2044 | Bacillus licheniformis | 0.998 | n.d. | n.d. |
| | 2045 | Bacillus galactosidilyticus | 0.990 | n.d. | n.d. |
| | 2049 | Bacillus oleronius | 0.990 | n.d. | n.d. |
| | 2052 | Rummeliibacillus pycnus | 0.970 | n.d. | n.d. |
| 2312 (Infected) | 2039 | Bacillus licheniformis | 0.982 | n.d. | n.d. |
| | 2047 | Lysinibacillus fusiformis | 0.970 | n.d. | n.d. |
| | 2048 | Sporosarcina contaminans | 0.980 | n.d. | n.d. |
| | 2050 | Streptococcus thoraltensis | 0.990 | n.d. | n.d. |
| | 2051 | Rummeliibacillus pycnus | 0.970 | n.d. | n.d. |
| | 3308 | Lactobacillus mucosae | 0.996 | n.d. | n.d. |
| 2373 (Infected) | 1063 | Escherichia coli | 0.987 (Shigella flexneri/ | − | n.d. |
| | | | Escherichia fergusonii) | | |
| 2429 (Infected) | 3227 | Staphylococcus warneri | 0.990 | n.d. | n.d. |
| | 3138 | Pediococcus acidilactici | 0.990 | n.d. | + |
| 2435 (Infected) | 1049 | Escherichia coli | 0.980 (Shigella flexneri/ | − | n.d. |
| | | | Escherichia fergusonii) | | |
| 2436 (Infected) | 1070 | Escherichia coli | 0.973 (Escherichia fergusonii) | − | n.d. |
| 2507 (Infected) | 1064 | Escherichia coli | 0.960 (Shigella flexneri) | SLT-I | n.d. |
| | 3180 | Streptococcus pluranimalium | 0.990 | n.d. | n.d. |
| | 2029 | Bacillus licheniformis | 0.995 | n.d. | n.d. |

^{(a)}% identity of partial 16S rDNA to type strain or closest relative; +: positive PCR results; −: negative PCR results; n.d.: data not determined
*Cow #2211-A and 2211-B represent two different animals that were assigned the same number at different times.

Pediocin Production

PCR screening revealed that Ped. acidilactici FUA3137, FUA3140, and FUA3138 harboured the pediocin AcH/PA-1 immunity gene (data not shown). Pediocin production was investigated via deferred inhibition assays, with the results depicted in FIGS. 5A and 5B. FIG. 5A depicts the results with no addition of proteinase, while FIG. 5B depicts the addition of proteinase K adjacent to colonies of test strains. Arrows indicate the site of proteinase K application. The following test strains were used: 1, Ped. acidilactici FUA3138; 2, Ped. acidilactici FUA3072; 3, Ped. acidilactici FUA3140; 4, Lact. sakei FUA3089. Similar results were observed with Listeria innocua ATCC33090 used as an indicator strain (data not shown). The indicator strains of E. coli FUA1036, FUA1063 and FUA1064 were also used but no inhibition was observed (data not shown).

Referring to FIG. 5A, Ped. acidilactici FUA3138 and FUA3140 produced inhibition zones against Enterococcus faecalis FUA3141 Inhibition zones of comparable diameter were observed with L. innocua (data not shown). Referring to FIG. 5B, further tests with proteinase K verified that the antimicrobial agent is a protein. Other vaginal isolates including E. coli FUA1036, FUA1063, and FUA1064 were also used as indicator strains but no inhibition was observed (data not shown). Test strains were grown on mMRS and overlayered with Enterococcus faecalis FUA3141, which was as an indicator strain.

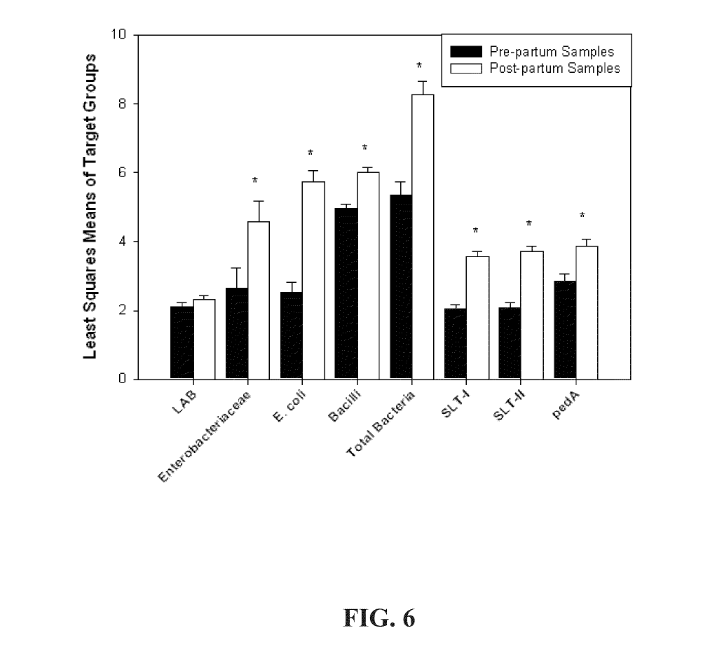

Quantification of bacterial groups, SLT and pediocin structural genes PCR-DGGE analysis was initially carried out characterize bovine vaginal microbiota by a culture-independent approach. The DNA concentration of samples from healthy cows, however, was below the detection limit of PCR-DGGE analysis and DGGE patterns could be obtained only for two samples obtained post partum (data not shown). A clone library that was later constructed using PCR products that were amplified with HDA primers from these two animals confirmed that all bacteria present in the bovine vagina were accounted for by culturing (data not shown). To overcome limitations of PCR-DGGE analysis, quantitative PCR was employed as sensitive and quantitative tool for culture-independent analysis of the composition of vaginal microbiota before and after parturition. Primers were selected to quantify bacterial groups isolated from healthy, pre-partum or postpartum animals, as well as SLT genes and the pediocin structural gene (pedA) (Table 6 and 3). Ten animals were sampled two weeks pre-partum and two weeks post-partum. To account for the large individual differences in the vaginal microbiota of different animals, results were calculated as differences (post-partum-pre-partum) between the least square means of log rDNA or DNA copy numbers for each target group. Referring to FIG. 6, differences in least squares means of log rDNA or DNA copy numbers of target groups are depicted. Vaginal mucus was sampled from ten animals before and after calving, and bacterial rDNA, shiga-like-toxin genes, and the pediocin structural gene were quantified by qPCR. The figure depicts the differences in least squares means of the target groups. Statistically significant differences between prepartum and postpartum periods were observed in all groups (as indicated by *) except for the lactic acid bacteria group. Number of rDNA copies of the Lactobacillus group relatively stable in the observation period with no statistically significant changes between the pre-partum and post-partum periods. In all other cases, the postpartum gene copy values are higher than the prepartum values. The pediocin structural gene was consistently detected in low numbers. Approximately a 3 log difference between the total bacteria values was observed. This increase was predominantly attributable to increased numbers of E. coli and Enterobacteriaceae. E. coli increased on average by more than 3 log. Genes coding for SLT-I and SLT-II increased by less than 2 log.

Discussion

This study provides a comparison of the vaginal microbiota of healthy, pregnant dairy cows, and infected postpartum cows by using culture, PCR, and qPCR. In contrast to the stable commensal microflora observed in humans and other mammals, total bacterial numbers in vaginal mucus were low and the composition of the bovine vaginal microbiota on species level was highly variable. Bacteria found within the microbiota are thus likely to be contaminants from the environment, the cow's skin, and or fecal material, rather than representing a stable flora autochthonous to the reproductive tract. Quantitative PCR confirmed the presence of lactic acid bacteria, staphylococci, E. coli, and bacilli in the vagina of pregnant dairy cows. Moreover, counts of Enterobacteriaceae and E. coli were found 1000 fold higher in infected, post-partum cows compared to samples from the same animals obtained pre-partum.

Overall, our data indicated that vaginal bacterial flora in cows affected by metritis was dominated by strains of E. coli, supporting previous observations. This study extends previous results by documenting changes of the vaginal microbiota in individual animals in the first two weeks after calving. Both the Enterobacteriaceae and E. coli showed marked increase in mucus samples collected from infected postpartum cows. The amplification of Shigella rDNA with E. coli species-specific primers is not surprising because Shigella spp. and E. coli are indistinguishable on the basis of rDNA sequences. In keeping with the recognition of Shigella spp. as human-adapted pathovar of E. coli, all isolates were identified as E. coli by biochemical tests. Culture-based analysis and qPCR demonstrated presence of shiga-like-toxin producing E. coli (STEC) in both healthy and infected animals. Three out of eleven E. coli isolates were found to carry genes coding for SLT-1 or SLT-II. Moreover, SLT-genes were consistently detected by qPCR in samples from metritic cows; STEC accounted for about 1-10% of the total E. coli population. SLT production causes diarrhoea in calves, but the role of STEC in the pathogenesis of metritis in adult animals warrants further clarification.

Bacilli are present in the environment and they frequently contaminate the bovine uterine lumen. However, pediococci have not yet been described as part of the bovine vaginal microbiota. The genus Pediococcus is closely related to the genus Lactobacillus. Pediococci produce antimicrobial compounds such as organic acids, hydrogen peroxide, and antimicrobial peptides such as pediocin AcH/PA-1. Ped. acidilactici are applied as starter cultures for meat fermentation and are additionally used as probiotic cultures, or as protective cultures to inhibit food-borne pathogens such as L. monocytogenes or Staphylococcus aureus. Ped. acidilactici was isolated from the gastrointestinal tract of poultry, ducks, and sheep and pediocin AcH/PA-1 producing strains have been isolated from human infant faeces.

The synthesis of pediocin AcH/PA-1 was initially described for the strains Ped. acidilactici PAC1.0 and Ped. acidilactici H, but synthesis has also been observed in other Ped. acidilactici strains as well as Lactobacillus plantarum WHE92, Pediococcus parvulus AT034, and AT077. Pediocin AcH/PA-1 production is a plasmid-borne trait. The pediocin AcH/PA-1 operon consists of pediocin AcH/PA-1 gene (pedA/papA), a specific immunity gene (papB), and genes responsible for processing and secretion (papC and papD). Pediocin production was confirmed for two isolates through sequencing and deferred inhibition assays as well as their ability to inhibit growth of Enterococcus faecalis and L. innocua. In keeping with prior reports on pediocin activity, pediocin was not active against E. coli, the dominant organisms in the vaginal microbiota of infected animals. A majority of pediocin producing isolates harboured the pediocin AcH/PA-1 operon, and qPCR analysis consistently detected the operon in both prepartum and postpartum vaginal samples.

Studies on the isolation of bacteriocin-producing lactic acid bacteria from the human vagina and their antimicrobial activities against human vaginal pathogens are well established. Bacteriocin-producing Lactobacillus strains inhibited vaginal pathogens including Gardnerella vaginalis and Pseudomonas aeroginosa. Although bovine vaginal microbiota have much lower total cell counts and lactobacilli populations in comparison to the human vaginal microbiota, bacteriocin such as pediocin may influence the microbial ecology in the reproductive tract of dairy cattle if bacteriocin-producing lactic acid bacteria are administered in high numbers.

### CONCLUSIONS

In conclusion, culture-dependent analysis of vaginal microbiota of dairy cows, supported by PCR and qPCR analyses, allowed the characterization of the bovine vaginal microbiota of healthy pregnant and infected postpartum cows and the identification of Shiga-like-toxin producing strains of E. coli. Identification of pediocin-producing pediococci in the bovine vaginal microbiota may encourage the development of novel prophylactic interventions against metritis by application of bacteriocin-producing probiotic bacteria into the vaginal tract of dairy cows during the transition period.

Further Discussion

In addition to the study described above, another test was performed in which one group of dairy cows received two treatments at −2 and −1 weeks before parturition, and another group of dairy cows received two treatments at −2 and −1 weeks before parturition and another treatment at +1 week after parturition. It was found that, while both groups of dairy cows exhibited benefits over the control group, the first group that only received treatment before parturition

Gene Accession Numbers of 16S rRNA Gene Sequences Obtained in this Study

Sequences of 16S rRNA genes of isolates obtained in this study were deposited in GenBank® with the following accession numbers: FUA3086 (GQ222397), FUA3087 (GQ222398), FUA3088 (GQ222399), FUA3089 (GQ222408), FUA1167 (GQ205673), FUA1035 (GQ222390), FUA1037 (GQ222410), FUA3137 (GQ222393), FUA3140 (GQ222392), FUA3141 (GQ222407), FUA3226 (GQ222394), FUA3136 (GQ205672), FUA1062 (GQ222401), FUA2027 (GQ205674), FUA2028 (GQ222400), FUA3251 (GQ222395), FUA1046 (GQ222387), FUA3135 (GQ222404), FUA2023 (GQ205670), FUA2024 (GQ205671), FUA1036, (GQ222389), FUA3139 (GQ222406), FUA1063 (GQ222403), FUA3227 (GQ205669), FUA3138 (GQ222409), FUA1049 (GQ222388), FUA1070 (GQ222391), FUA1064 (GQ222405), FUA3180 (GQ222402), FUA2029 (GQ222396).

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

The following claims are to be understood to include what is specifically illustrated and described above, what is conceptually equivalent, and what can be obviously substituted. The scope of the claims should not be limited by the preferred embodiments set forth in the examples, but should be given the broadest interpretation consistent with the description as a whole.

What is claimed is:

1. A method of using probiotic bacterial strains as a prophylactic tool against uterine infections in a pregnant female ruminant, comprising the step of:
prior to parturition, intravaginally administering the female ruminant with a sufficient count of one or more bacterial strains effective for treating pathogenic microorganisms associated with postpartum infection of the uterus of female ruminants.

2. The method of claim 1, wherein the female ruminant is a female bovine.

3. The method of claim 1, wherein the pathogenic microorganisms comprise Actinomyces pyogenes, Escherichia coli, Fusobacterium necrophorum, and Bacteroides melaminogenicus.

4. The method of claim 1, wherein the one or more bacterial strains comprise Lactobacillus sakei FUA 3089, Pediococcus acidilactici FUA 3140, Pediococcus acidilactici FUA 3138, or combinations thereof.

5. The method of claim 1, wherein the one or more bacterial strains comprise one or more bacteriocin-producing lactic acid bacterial strains.

6. The method of claim 1, wherein the one or more bacterial strains comprise a bacterial count of at least 10¹⁰ cfu.

7. The method of claim 1, further comprising the step of administering the one or more bacterial strains at intervals prior to parturition.

8. The method of claim 1, wherein the bacterial strains are only administered prior to parturition.

9. A method of using probiotic bacterial strains as a prophylactic tool against uterine infections in a pregnant female ruminant, comprising the step of:
prior to parturition, intravaginally administering the female ruminant with one or more of the bacterial strains Lactobacillus sakei FUA 3089, Pediococcus acidilactici FUA 3140, and Pediococcus acidilactici FUA 3138.

10. The method of claim 9, wherein the female ruminant is a female bovine.

11. The method of claim 9, wherein the one or more bacterial strains comprise a bacterial count of at least 10⁸ cfu.

12. The method of claim 9, wherein the one or more bacterial strains comprise a bacterial count of at least 10⁹ cfu.

13. The method of claim 9, wherein the one or more bacterial strains comprise a bacterial count of at least 10¹⁰ cfu.

14. The method of claim 9, further comprising the step of administering the one or more bacterial strains at intervals prior to parturition.

15. The method of claim 9, wherein the bacterial strains are effective for preventing pathogenic microorganisms from causing postpartum infection of the uterus of female ruminants

16. The method of claim 15, wherein the pathogenic microorganisms comprise at least one of Actinomyces pyogenes, Escherichia coli, Fusobacterium necrophorum, and Bacteroides melaminogenicus.

17. The method of claim 9, wherein the bacterial strains are applied only prior to parturition.

18. A method of using probiotic bacterial strains as a prophylactic tool in a pregnant female ruminant, comprising the step of:
prior to parturition, intravaginally administering the female ruminant with bacterial strains comprising one or more of the bacterial strains Lactobacillus sakei FUA 3089, Pediococcus acidilactici FUA 3140, and Pediococcus acidilactici FUA 3138.

19. The method of claim 18, wherein the female ruminant is a female bovine.

20. The method of claim 18, wherein the one or more bacterial strains comprise a bacterial count of at least 10⁸ cfu.

21. The method of claim 18, wherein the one or more bacterial strains comprise a bacterial count of at least 10⁹ cfu.

22. The method of claim 18, wherein the one or more bacterial strains comprise a bacterial count of at least 10¹⁰ cfu.

23. The method of any of claim 18, further comprising the step of administering the bacterial strains at intervals prior to parturition.

24. The method of any of claim 18, wherein the bacterial strains are effective for preventing pathogenic microorganisms from causing postpartum infection of the uterus of female ruminant

25. The method of claim 24, wherein the pathogenic microorganisms comprise Actinomyces pyogenes, Escherichia coli, Fusobacterium necrophorum, and Bacteroides melaminogenicus.

26. The method of claim 18, wherein the bacterial strains are applied only prior to parturition.

27. A method of using probiotic bacterial strains as a prophylactic tool in a pregnant female ruminant, comprising the step of:
prior to parturition, intravaginally administering the female ruminant with one or more bacteriocin-producing lactic acid bacterial strains.

28. The method of claim 27, wherein the female ruminant is a female bovine.

29. The method of claim 27, wherein the one or more bacterial strains comprise a bacterial count of at least 10⁸ cfu.

30. The method of claim 27, wherein the one or more bacterial strains comprise a bacterial count of at least 10⁹ cfu.

31. The method of claim 27, wherein the one or more bacterial strains comprise a bacterial count of at least 10¹⁰ cfu.

32. The method of claim 27, further comprising the step of administering the bacterial strains at intervals prior to parturition.

33. The method of claim 27, wherein the bacterial strains are effective for preventing pathogenic microorganisms from causing postpartum infection of the uterus of female ruminants

34. The method of claim 33, wherein the pathogenic microorganisms comprise at least one of Actinomyces pyogenes, Escherichia coli, Fusobacterium necrophorum, and Bacteroides melaminogenicus.
